(19) United States
(12) Patent Application Publication
(10) Pub. No.: US 2022/0117543 A1
(43) Pub. Date: Apr. 21, 2022

(54) Mucosal Impedance Measuring Device With Endoscopic Articulation

(71) Applicant: Diversatek Healthcare, Inc., Milwaukee, WI (US)

(72) Inventor: Charles Lindsay, Monument, CO (US)

(21) Appl. No.: 17/434,337

(22) PCT Filed: Feb. 25, 2020
(86) PCT No.: PCT/US2020/019702

§ 371 Date: Aug. 26, 2021

Related U.S. Application Data
(60) Provisional application No. 62/812,034, filed on Feb. 28, 2019.

(51) Int. Cl. A61B 5/00; A61B 5/0538; A61B 1/005; A61B 1/273

(52) U.S. Cl. CPC A61B 5/4233; A61B 5/687; A61B 1/2733; A61B 5/6852; A61B 1/0051; A61B 5/0538

(57) ABSTRACT

A mucosal impedance measuring device measures a pressure-controlled impedance of mucosal tissue. The device includes an endoscope having an elongated body extending to a scope end in which the scope end is articulable relative to the elongated body and a plurality of impedance measuring electrodes supported by the endoscope proximate the scope end. Upon articulation of the scope end, the plurality of impedance measuring electrodes are moved relative to the elongated body to be drawn into contact with mucosal tissue under an applied pressure to collect pressure-regulated impedance of the tissue. In some forms, an impedance measuring system can be in electrical communication with the electrodes and may include software that determines whether the pressure-controlled impedance of the mucosa that is measured is a stable impedance measurement indicative of consistent pressure-regulated contact between the impedance measuring electrodes and the mucosa. Still further, such device may be used to map and provide a visual indication of the colon or other portion of the gastrointestinal tract and visually provide impedance measurements associate with one or more regions of the gastrointestinal tract.

### CROSS-REFERENCE TO RELATED APPLICATIONS

This application claims the benefit of U.S. Provisional Patent Application No. 62/812,034 entitled “Mucosal Impedance Measuring Device with Endoscopic Articulation” filed Feb. 28, 2019, which is hereby incorporated by reference for all purposes as if set forth in its entirety herein.

### STATEMENT REGARDING FEDERALLY SPONSORED RESEARCH

Not applicable.

### BACKGROUND

Recently, links between the electrical properties of esophageal mucosal tissues and the health of these esophageal mucosal tissues have been identified and investigated. Specifically, it has been determined that by the taking impedance measurements of the esophageal mucosa, that the condition or health of those esophageal mucosa can be determined. For example, patients with gastroesophageal reflux disease (GERD) have damaged mucosa in regions of their esophagus that are evident when the impedance measurements of the damaged mucosa are compared to the measurements of healthy, undamaged mucosa. Importantly, such impedance measurements are taken with the impedance measuring electrodes being placed in controlled contact with the esophageal mucosa under regulated pressure. By ensuring good pressure contact between the electrodes and the esophageal mucosa, meaningful readings can be obtained which closely correspond to the health of the tissues based on empirical data.

Understanding this relationship between the impedance measurements of the esophageal mucosal and the health of the esophageal mucosal, unique devices have been designed to take such health-indicating impedance measurements. For example, U.S. Pat. No. 9,814,408 issued on Nov. 14, 2017 and U.S. Patent Application Publication No. 2017/0143248 published on May 25, 2017, both of which are incorporated by reference for all purposes as if set forth in their entirety herein, disclose systems and methods involving the use of catheters and catheter systems that take pressure regulated impedance measurements of esophageal mucosa. Such devices are described as, for example, including catheters with associated balloons and impedance measuring electrodes which might be arranged in arrays or lines. During use of such a device, the patient is intubated with the catheter, the balloon is inflated to draw the electrodes into predicable and controlled contact with the esophageal mucosa, and impedance measurements are taken through the electrodes while the balloon remains inflated. The measured impedance can then be evaluated and compared to the known impedance values of heathy or unhealthy tissue.

Such devices are contemplated as permitting much faster identification of esophageal conditions as opposed to more traditional studies, which can last for multiple hours or even a few days.

### SUMMARY OF THE INVENTION

While catheterized systems of the type described above have been demonstrated to be able to collect useful impedance measurements of esophageal mucosa for time-efficient evaluation of esophageal mucosal health, these systems still are somewhat limited in their application.

For instance, while a balloon catheter may be viable for evaluating the tissue of an esophagus of a patient, the structure of the catheter and its associated balloon limits the applicability of the system. If there were other ways of locally measuring impedance without using, for example, an inflatable balloon, then this technology might be more widely applied to the study and interrogation of other types of mucosa as well, such as mucosa in the latter portions of the gastrointestinal tract. The use of a balloon catheter in that potential environment (i.e., the lower portions of the gastrointestinal tract) is simply not believed viable given the tortious path of the gastrointestinal tract.

Still further, while catheterized devices may be relatively well adapted for the esophagus, positioning the electrodes in the places of greatest interest can remain a challenge, sometimes requiring the use of pull-out techniques, in which the catheter is inflated in a first location, measurements taken, the catheter deflated and withdrawn a small distance, and then re-inflated and more measurements taken.

Disclosed herein are improvements, including new devices and methods, of measuring mucosal impedance with wider adaptability and applicability. Herein, it is proposed to take an endoscope and provide the impedance measuring electrodes on the scope end of the endoscope. When it is desired to collect an impedance measurement of the surrounding mucosa, the scope end of the endoscope is articulated relative to the rest of the endoscope to press the impedance measuring electrodes disposed on that articulated end against the mucosa. At this point, impedance measurements of the mucosa can locally be taken, and an attached impedance measurement system collecting these measurements can assess or review the collected impedance measurements to see whether the collected impedance measurements are taken under stable conditions (i.e., under consistently good contact with the mucosa) as impedance measurements taken under low or unsteady pressure may not be appropriately reflective of the condition of the mucosa. Such devices may use hardware or software in the impedance measuring device to access the impedance measurement and determine whether it is reflective of a good, viable measurement based on a quality (for example, whether the measurement is sufficiently stable over a pre-determined time duration) or whether it is reflective of a bad measurement that should be disregarded (because, for example, it is too variable over a predetermined duration of time or has an absolute value that is outside of an expected range of expected values regardless of whether the mucosa are healthy or not). Still further, by placement of the impedance measuring electrodes on the scope end of an endoscope, the placement of the electrodes on the tissue may be done with a visual interrogation might be performed simultaneously or at least in close proximity in time with the electrical interrogation of the tissue. Given this design, it is believed that such devices and methods might be applicable to mucosal studies extending beyond just the mucosa of the esophagus and would be well suited, in particular, to studies in the lower portions of the gastrointestinal tract which also have mucosa which before this time have not been widely characterized by their impedance.

According to one aspect, a mucosal impedance measuring device is provided for measuring a pressure-controlled impedance of mucosa. The device includes an endoscope having an elongated body extending to a scope end in which the scope end is articulable relative to the elongated body. The device further includes a plurality of impedance measuring electrodes supported by the endoscope proximate the scope end. Upon articulation of the scope end, the plurality of impedance measuring electrodes are moved relative to the elongated body to be drawn into contact with the mucosa under an applied pressure from the articulation.

In some forms, the device may further include an impedance measuring system in electrical communication with the plurality of impedance measuring electrodes in which the impedance measuring system is configured to direct a current between the plurality of impedance measuring electrodes and through the mucosa and is further configured to measure the pressure-controlled impedance of the mucosa. The impedance measuring system may include software and/or hardware configured to determine whether the pressure-controlled impedance of the mucosa is a stable impedance measurement indicative of consistent pressure-regulated contact between the plurality of impedance measuring electrodes and the mucosa. Even though the validity of a taken measurement may happen automatically (i.e., every taken measurement may be assessed for validity), it is still contemplated that the articulation of the scope end of the endoscope may be manually controlled by an operator as well as the request to electrically interrogate the tissue may be manually instructed by an operator.

In some forms, the device may further include a support attachable to the endoscope proximate the scope end with the support supporting the plurality of impedance measuring electrodes thereon. For example, the support may be a sleeve that at least partially (or wholly) surrounds the circumference of the elongated body of the endoscope at the scope end. When there is a support or sleeve element, it is contemplated that it may take on various geometries. For example, the support might be tubular and/or compressively received around the scope end of the endoscope. However, in other forms, the support might be C-shaped—effectively a sleeve with an axially extending slit—and only partially encircle the circumference of the endoscope. Still yet, if there is a support it might be attached in other ways such as by fastening structures and/or adhesive engagement, for example.

In some forms, it is contemplated that the impedance measuring electrodes might be directly connected to the scope end of the endoscope with any kind of intermediate support. In such designs, the electrodes would then be integrated into the overall scope design.

In some forms, the mucosal impedance measuring device may further include a plurality of conductors (e.g., wires or leads) in which each of the conductors are in electrical communication with a corresponding one of the plurality of impedance measuring electrodes and in which the conductors extend from the impedance measuring electrodes. It is contemplated that, in some forms, the plurality of conductors may be received in a working channel of the endoscope; however, in other forms, the conductors may run along the outside of the endoscope and be separate therefrom. The latter may be preferable when the number of conductors is high and/or the working channel of the endoscope cannot accommodate the size of the bundle of conductors.

In some forms, the conductors may extend to a connector on an end of the conductors opposite their attachment to the impedance measuring electrodes at the scope end. It is contemplated that this connector may include a plurality of axially-spaced contacts with each of the plurality of axially-spaced contacts being in electrical communication with one of the plurality of conductors and thereby in electrical communication with a corresponding one of the plurality of impedance measuring electrodes. The connector may also be roughly cylindrical in shape and not larger in diameter than the working channel in order to permit passage therethrough during assembly. Such a conductor and connector arrangement is particularly advantageous in the context of endoscopes, because the conductors are to be kept small in diameter to be viable given the small size of the working channel (e.g., less than 2.8 mm in diameter) as opposed to harness-type connectors would not be viable using the working channel without complex assembly of the connector to the conductor bundle after the conductors have been passed through the working channel.

According to another aspect, a method is disclosed of measuring a pressure-controlled impedance of mucosa. A scope end of an endoscope is articulated relative to an elongated body of the endoscope to draw a plurality of impedance measuring electrodes on the scope end of the endoscope into contact with mucosa under an applied pressure from the articulating. After articulation and while continuing to hold the electrodes against the mucosa, a current is conducted between the plurality of impedance measuring electrodes and through the mucosa and the pressure-controlled impedance of the mucosa is measured.

In some forms, the step of measuring the pressure-controlled impedance may involve using an impedance measuring system in electrical communication with the plurality of impedance measuring electrodes. The impedance measuring system may direct the current between the plurality of impedance measuring electrodes and through the mucosa and measure the pressure-controlled impedance of the mucosa.

In some forms, the method may further include determining, using the impedance measuring system, whether the pressure-controlled impedance of the mucosa is a stable impedance measurement indicative of consistent pressure-regulated contact between the plurality of impedance measuring electrodes and the mucosa. Again, it is contemplated this determination step might be performed by software and/or hardware of the impedance measuring system. Still yet, it will be appreciated that, while this determination step may occur automatically upon the taking of an impedance measurement, the step of articulating the scope end of the endoscope and instructing the impedance be measured may still be performed manually by an operator. Accordingly, while the device is largely controllable by the operator, when a measurement is attempted, the system is capable of providing feedback regarding the quality of the measurement taken (e.g., whether the measurement taken a valid one based on contact and/or stabilization of the impedance measurement over time or not).

In some forms, the method may further involve the step of attaching a support proximate the scope end in which the support includes the plurality of impedance measuring electrodes thereon. It is contemplated that, in some forms, the support may be compressively connected to the scope end of the endoscope although the support could again take many different geometric forms. Still further, as mentioned above, the impedance measuring electrodes and/or the conductors might be integrated into the endoscope itself in some designs.

According to still another aspect, a method of visually indicating a portion of a gastrointestinal tract is provided. One or more spatial locations of an endoscope are recorded during an insertion of the endoscope into and through the gastrointestinal tract. A location of the endoscope is visually depicted within a generated image of the gastrointestinal tract.

In some forms, the method may further include the steps of articulating a scope end of an endoscope relative to an elongated body of the endoscope to draw a plurality of impedance measuring electrodes on the scope end of the endoscope into contact with mucosa under an applied pressure from the articulating, conducting a current between the plurality of impedance measuring electrodes and through the mucosa, and measuring the pressure-controlled impedance of the mucosa. The pressure-controlled impedance of the mucosa is visually indicated on the generated image of the gastrointestinal tract at a location at which the pressure-controlled impedance of the mucosa is taken. In some cases, the pressure-controlled impedance of the mucosa are repetitively collected at various locations in the gastrointestinal tract. The pressure-controlled impedance of the mucosa is visually depicted at various locations in the gastrointestinal tract on the generated image of the gastrointestinal tract corresponding to the locations at which each respective pressure-controlled impedance of the mucosa is taken.

In some forms, the step of visually indicating the pressure-controlled impedance of the mucosa on the generated image may involve associating and displaying a color associated with a value (e.g., a range of values) of the pressure-controlled impedance in a region associated the location at which the pressure-controlled impedance is taken.

These and still other advantages of the invention will be apparent from the detailed description and drawings. What follows is merely a description of some preferred embodiments of the present invention. To assess the full scope of the invention, the claims should be looked to as these preferred embodiments are not intended to be the only embodiments within the scope of the claims.

### BRIEF DESCRIPTION OF THE FIGURES

FIG. 1 is a schematic side view of a mucosal impedance measuring device showing a support or sleeve that carries a plurality of impedance measuring electrodes and that is received on the scope end of an endoscope. Note that as this is an exemplary schematic all details of the endoscope are not illustrated such as the camera, articulation mechanism, and so forth.

FIG. 2 is a schematic side view of the device of FIG. 1 being articulated to bring the electrodes of the device in contact with mucosa for taking an impedance measurement.

FIG. 3A is an axial view of the sleeve carrying the electrodes from FIG. 1 apart from the endoscope.

FIG. 3B is a side view of the sleeve of FIG. 3A.

FIG. 4 is an enlarged side view of a connector which may be found at the end of the conductors connected to the impedance measuring electrodes opposite the support/sleeve end.

FIG. 5 is a schematic illustration of an example system setup in which the impedance measuring device including the impedance measuring system and impedance measuring electrodes are apart from the endoscope and its associated control system.

### DETAILED DESCRIPTION

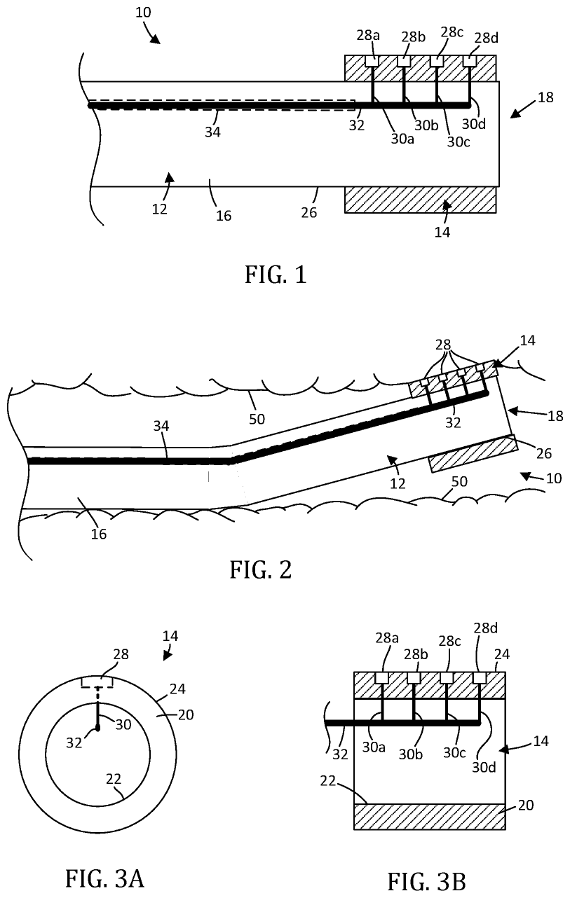

Referring to FIGS. 1 through 5, a mucosal impedance measuring device 10 and its constituent parts are illustrated. As illustrated, the mucosal impedance measuring device 10 includes an endoscope 12 and a support 14 attached to the endoscope 12 which serves as an accessory for electrical interrogation of mucosa as will be described in greater detail below. It is noted that, while the endoscope 12 and the support 14 are illustrated as separate components in the illustrated embodiment, it is contemplated that the accessory structure of the support 14 might in other forms be integrated directly into the structure of an endoscope and such combined structure is contemplated under the scope of this disclosure rather than the illustrated multi-component construction.

As illustrated in FIGS. 1 and 2, the endoscope 10 has an elongated body 16 extending to a scope end 18. Such an endoscope as endoscope 10 is generally known from the state of the art and the scope end 18 typically includes a camera and light (both not shown in the schematic illustration) on the axial end thereof for producing images of the inside of a patient or subject. The scope end 18 is articulable relative to the rest of the elongated body 16. This articulation both can be used to guide the endoscope 12 as it is inserted into the patient or subject and to direct the camera on the scope end 18 to capture video and/or images of interest within the body. The control of the articulation as well as the insertion/withdrawal of the endoscope 10 is typically manually performed by an operator such as a physician or technician to ensure that the scope end 18 is navigated appropriately.

Referring now to FIGS. 3A and 3B, the support 14 is shown separately from the endoscope 12. In the form shown, the support 14 is a generally tubular sleeve 20 having a radially-inward facing surface 22 and a radially-outward facing surface 24 that can be received on the scope end 18 of the endoscope 12. Such a support 14 may be relatively short relative to the overall length of the endoscope 12. For example, it is contemplated that the support 14 may be approximately 2 cm long in some forms.

To provide mechanical attachment between the two, the support 14 can be made of, for example, a polymeric or elastomeric material and sized such that the radially-inward facing surface 22 is approximately the same size or smaller than a radially-outward facing surface 26 of the scope end 18 of the endoscope 12. In such case, with a small amount of temporary deformation of the body of the support 14, the support 14 can be compressively secured to the scope end 18 of the endoscope 12 by axial insertion thereon. Of course, this is but one way of attaching the support 14 to the scope end 18 of the endoscope 12 and, in constructions in which there is a support other modes of attachment might also be used. For example, mechanically interlocking parts such as snaps or bayonet-type connections might be used to create mechanical engagement or fasteners of other types might form a connection between the two components. Still further, an adhesive, epoxy, or resin, whether permanent or temporary may be used to similar effect.

Notably, the support 14 carries a plurality of impedance measuring electrodes 28a-d (generally, indicated in some views by the number “28” alone) which are exposed on the radially-outward facing surface 26 of the support 14. These impedance measuring electrodes 28a-d are each attached to a separate and unique conductor 30a-d (generally referred to as “30” and which can be, for example, wires) which can then grouped into a bundle 32 which is received in the working channel 34 of the endoscope 12. While the conductors 30a-d and the bundle 32 are illustrated as going directly into the endoscope 12 and the working passage 34 thereof, it will be appreciated that this is for ease of understanding and depiction. In reality and based on the structure of the endoscope 12, the conductors 30a-d and the bundle 32 may be otherwise situated or routed, for example, first extending axially forward to the front of the scope end 18 and then wrapping back into the working passage 34 of the endoscope 12. Of course, it is also possible that the endoscope may be adapted in some forms to permit the passage of the conductors or bundle through the sidewall by the presence of an axially extending slit for instance.

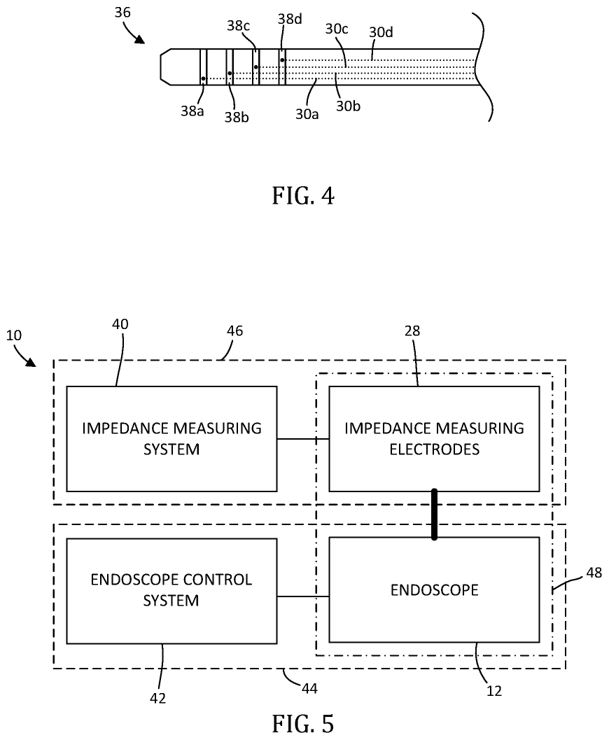

On the end of the endoscope 12 opposite the scope end 18, and as illustrated in FIG. 4, the various conductors 30a-d of the bundle 32 may then be adapted for a connector 36 which has various axially-spaced connector contact rings 38a-d which may be placed into electrical communication with an impedance measuring system 40 as will be described in greater detail below. Notably, because space is at a premium in an endoscope, a typical working channel 34 is very small (for example, only 2.8 mm in diameter) and so there is not much space for either a connector 36 of the bundle 32 to pass to when the support 14 is attached to the endoscope 12 and the connector 36 and bundle 32 threaded through the working channel 34 of the endoscope 12. Accordingly, a narrow, cylindrical profile for the connector 36 is advantageous in accommodating assembly.

As illustrated, the support 14 includes a line of four impedance measuring electrodes 28a-d which are axially spaced over the length of the support 14; however, other configurations are contemplated. For example, there may be more or less than four impedance measuring electrodes or the impedance measuring electrodes may be differently situated on the support (e.g., they do not all need to be all in an axial line or there may be multiple electrodes positioned at differing angular positions). Further, it is contemplated that the electrodes could be place on various sides or faces of the sleeve such that, regardless of the manner of endoscopic articulation, some of the electrodes would be placed in contact with mucosa. In this configuration, it may be possible to take various mucosal impedance measurements around the periphery of the tissue at a particular insertion depth or position of the endoscope to collect additional samples/measurements at a location. Still further, it is contemplated that the electrodes might be ringed-shaped around the periphery or outer circumference of the sleeve, although some consideration may need to be made for the amount of surface area of the ring electrodes contacting the mucosa.

It is also contemplated that the bundle of conductors might run parallel to the endoscope on the exterior of the endoscope and that the bundle and endoscope may be, for example, retained in a protective sheath to keep the two together. This exterior configuration might be particularly advantageous when there are a large number of sensors.

With additional reference now being made to FIG. 5, it can be seen how the impedance measuring device 10 can include an impedance measuring system 40 which is placed into electrical communication with the impedance measuring electrodes 28 (for example, by plugging in the connector 36 into the impedance measuring system 40). The impedance measuring system 40 can include a processor, a current source, and circuitry for measuring the impedance between the impedance measuring electrodes 28. For example, it can include a current production source which can be used to run current from one electrode to another (and through mucosa, as will be described below) and circuitry for measuring the impedance between the electrodes (and, again, of the mucosa therebetween).

It is contemplated that the impedance measuring system 40 can not only include electronics for obtaining impedance measurements, but also software and/or hardware for determining whether the measured impedance measurements are valid. Since, as explained above and from the patent and application incorporated by reference, the impedance measurements of mucosa are only valid if taken under controlled pressure and are sufficiently stable (meaning that good consistent contact is made between the electrodes and the mucosa), it is contemplated that the impedance measuring system 40 can include testing logic to evaluate and confirm with the end user whether an obtained impedance measurement of mucosa is a good and valid measurement or includes stability issues or absolute impedance values that are indicative of an improper reading due to bad or inconsistent contact between the electrodes and the mucosa. For example, it is contemplated that impedance might be measured over a predetermined window of time (perhaps, a few hundred milliseconds or various seconds) and the signal of the impedance measured over time analyzed to determine whether the impedance is stable and within expected ranges for either healthy or unhealthy mucosa.

Still further, it is contemplated that there could be pressure-sensing elements attached to electrodes or between the electrodes that independently and electronically confirm stable pressure contact exists between the electrodes and mucosa when the scope end is articulate. Such pressure-sensing elements and information therefrom might be used separately from or in combination with the software/hardware analysis of the impedance signal to assess the validity of an impedance measurement.

FIG. 5 also highlights another aspect of the disclosed impedance measuring system 10—that, as illustrated, the support 14 containing the impedance measuring electrodes 28 is an add-on accessory for a pre-existing endoscope 12. In the disclosed configuration, the endoscope 12 is controlled by a separate endoscope control system 42 to define an overall endoscopic system 44. The impedance measuring system 40 along with the impedance measuring electrodes 28 (and any associated conductors, connectors, and so forth) are part of a separately controlled accessory system 46. The separately controlled accessory system 46 runs in parallel with the endoscopic system 44, and based on the physical connection of the impedance measuring electrodes 28 to the scope end of the endoscope 12 (as denoted by box 48 and the bold connection line), physically leverages the endoscope in operation because, for example, the accessory system 46 is navigated and placed based on the location of the endoscope 12 and then can operated independently thereof once it is in place.

However, it is also contemplated that rather than constructing and operating the mucosal impedance measuring device as part of a dual system configuration, that the endoscope might be designed with the mucosal impedance measuring device as being unitary with the endoscope. For example, rather than having a support as in the illustrated embodiment, the impedance sensing electrodes might be integrated into the endoscope itself. As such, the conductors might be more tightly integrated with the construction of the endoscope and need not utilize the working channel or run external to the endoscope as described above.

Having described the general structure of the mucosal impedance measuring device 10, a method of operation of this device will now be described, with reference to FIG. 2 being made in particular. In order to measure the impedance of mucosa 50 using an endoscope device such as that contemplated herein, the endoscope 12 is first inserted and navigated to the desired location within the patient. The camera of the endoscope 12 can then be used to determine that the endoscope 12 is generally placed within the patient as desired at the location where an impedance measurement of mucosa is desired. At this point, the scope end 18 of the endoscope 12 is articulated relative to the body 16 of the endoscope 12 in order to place the impedance measuring electrodes 28a-d into contact with the mucosa 50. The articulation itself applies the pressure to create the contact between the electrodes 28a-d and the mucosa 50. Upon articulation and continuing to be held in this articulated position to maintain contact between the mucosa 50 and the electrodes 28a-d, a current is conducted between the plurality of impedance measuring electrodes 28a-d and through the mucosa 50 and the pressure-controlled impedance of the mucosa is measured. The providing of the current and the measurement of the impedance of the mucosa 50 can all be performed by the impedance measuring system 40 which is external to the patient, but in electrical communication with the electrodes 28a-d via the conductors 30a-d. Notably, because the contact between the electrodes 28a-d and the mucosa 50 could potentially be insufficient for collecting a good measurement of the mucosa 50 depending on the particular placement of the scope and the surrounding mucosa, as previously mentioned the impedance measuring system 40 may seek to validate that the obtained measurement is accurately depicting the impedance of the mucosa 50. To this end, the impedance measuring system 40 can assess whether the particular reading appears to be good based on various pre-determined conditions and provide a good/bad or green/red indication to the operator regarding the quality of the taken impedance measurement. For example, if the absolute values are outside of the range of impedances expected for mucosa—suggesting improper contact is being made—then such readings may be so indicated as being bad or potentially invalid. Likewise, if the impedance measurements over time do not remain steady, then the impedance measuring system may either indicate that the impedance is not steady or stable and therefore is invalid or may be capable of resolving that unstable reading into a useable measurement based on certain qualities (for example, if the reading remains stable over certain periods of time, it may be determined that this is the proper and full contact measurement).

Such methods may also be employed in pull-out studies in which a reading is taken and then the device is pulled out some distance before taking another reading. Such pull-out studies permit a length of tissue to be mapped over distance in excess of the overall length of the electrodes on the device by taking and combining various measurements at known positions iteratively.

Still further, it is contemplated that the mucosal impedance measuring device 10 may be implemented in such a way that it can be used to map the passageway it is inserted into, specifically in the case of the gastrointestinal tract or colon, and display visual information relating to the gastrointestinal tract or colon dimensionally, to the location of the endoscope (particularly the scope end) within the gastrointestinal tract or colon, and/or to impedance measurements that have been taken relating to tissue health. Such visual depiction or display can occur on a monitor or other viewing device attached to the mucosal impedance measuring device and/or the endoscope. For example, during an insertion of the endoscope into and through the gastrointestinal tract, one or more spatial locations of an endoscope may be recorded. This recording of this positional data may be done manually for example, by the operator of the endoscope inputting information about the position of the scope end of the endoscope (i.e., providing the software information about when initial insertion is occurring, when the scope is at a bend between one region and another or at some other predefined location, and so forth). Such recording may be automated in part or in whole for example by software that prompts the operator for input of certain information or that monitors and analyzes the manner in which the endoscope is inserted and the manner in which is navigated through the passageways of the patient to detect these conditions in a “smart” manner. In some instances, it is contemplated the position determinations may be made in whole or in part using imaging from the camera or other modes of interrogation to determine the position and path of the scope end of the endoscope as it is inserted. With this positional information available, a location of the endoscope may be visually depicted within a generated image of the gastrointestinal tract produced by the method. For example, the operator or physician performing the endoscopy may map the interior of a colon during an initial insertion process by entering information about certain data points (e.g., when a curve from one region to another region of the colon is being made) which may be depicted on a computer monitor or display, for example. With the colon physically mapped and depicted, the operator will then be able to visually see the location of the scope end of the endoscope during further examination as the software accounts for the length of insertion or withdrawal of the endoscope once the mapping has occurred and presents this position on the mapped image.

It is contemplated that such visual depiction might be two dimensional or even three dimensional. Three dimensional depiction may require some additional input stream, such as potentially a video stream that calculates diameter of the patient's passageway locally, or may involve some other reading collected from the endoscope indicative of diameter of the colon in the localized region.

Still further, all of the mucosal impedance collecting steps described above with respect to the general operation of the mucosal impedance measuring device 10 may be performed contemporaneously with mapping or after mapping. Such mucosal impedance collection may be used to map a single point or multiple points (perhaps involving a length) of the colon with respect to health of the tissue. Indeed, when electrodes are accessible on different sides of the endoscopic tip, it may even be possible to take multiple peripheral measurements at a particular insertion depth of the endoscope for three dimensional inspection of the tissue. While the general understanding in the state of the art is that tissue at a particular insertion depth should be equally healthy on all sides, multiple readings may be able to be made to collect various data points which can then be averaged and/or used to determine whether there is a difference in tissue over the periphery that may be of interest.

Accordingly, such visualization of the gastrointestinal tract may include not just information about the physical dimensions of the gastrointestinal tract, but also provide indications of tissue health from the mucosal impedance measurements. After one or more collected mucosal impedance measurements are taken they then may then be visually mapped on corresponding region(s) of the map of the colon, for example. By visually plotting the mucosal impedance measurement(s) on a visual depiction of the colon, it may be easier for the operator or patient to visualize comprehend where the readings are being taken and, in the case of multiple readings, visualize more holistically the health of the tissue over a length of interest of the colon and understand the nature of any irregularity by providing better physical context, depicted visually.

When graphically depicting the mucosal impedance on the visual depiction of the gastrointestinal tract, it is considered that the impedance readings may be color-coated to improve understanding by the viewer. For example, measurements that are taken as being indicative of healthy tissue may be depicted as green, while unhealthy tissue depicted as red. Still further, color gradients could be used to depict either the magnitude of the reading (e.g., light green for marginally healthy tissue and dark green for strongly healthy tissue.

Still further, it is contemplated that apart from merely mapping tissue health using color and/or number, it is possible that quality of the reading (e.g., the stability of the reading) might also be mapped physically on the visualized depiction. In this way, the operator may also be able to better assess whether certain regions we more difficult than others to collect measurements from and, as a result of this difficultly in collection, if the measurements from that area may need to be recollected or be examined more closely or skeptically. Still further, it may be possible to provide other layers not just for health of tissue base on impedance or quality of reading, but for other conditions of interest, such as for example the average diameter of the localized region of the colon if such information has been collected.

Returning now to the structure of the endoscope and accessory for the endoscope, it is contemplated that in addition to the structures and methods described above, various other modifications and additions might be feasible to facilitate authentication of the device and/or ensure proper use. As one example, when the device is provided as an accessory to an endoscope, the accessory structure might house a small chip (for example, in the structure of the support) which could provide authentication information to the impedance measuring system to confirm that the accessory device is genuine and/or provide calibration information relating to the specific accessory so that, when that calibration information is accounted for by the impedance measuring system, the impedance measurements taken with the accessory are accurate. Still further, in such case that the impedance measuring electrodes are tightly integrated into the endoscope, such authentication information and/or calibration data might similarly be housed in the structure of the endoscope. It is also contemplated that such a chip or memory could store information about the number of times that the accessory (or endoscope, if integrated) has been used to ensure both that the accessory (or endoscope) is being properly used and has not been fouled in some way since its calibration. For example, the device may be engineered for one-time use or N-time uses and the chip may hold information about whether the use or N-time uses has occurred or not; it is contemplated that, if the use or uses have occurred, then the impedance measuring system may provide the user with an indication that the device cannot be used without first replacing the accessory (or endoscope, if integrated).

Thus, a structure is provided that allows for the collection of mucosal impedance measurements without inflatable elements. Among other things, it is contemplated that the structure disclosed herein can enable collection of mucosal tissue in locations—such as the lower portions of the gastrointestinal tract (e.g., in the colon) in which a balloon catheter may not be useable based on a tortuous path and for potentially having irregular and unexpected profile. Furthermore, it contemplated that this device and its use might prove to be a viable substitute for more involved or more invasive interrogations of the body and used to, for example, to evaluate tissue condition or tissue health such as, for example, to examine for colitis. It is contemplated that obtaining gastrointestinal impedance measurements might replace more invasive operations, such as the performing of a biopsy, which carries with it the risk of complication and potentially infection. Of course, nothing would preclude such an endoscope with such impedance measuring electrodes from being used in esophageal studies, where balloon-type catheters are also viable study tools. A further potential advantage of the disclosed structures and methods is that, by attachment to or integration of the impedance measuring electrodes with an endoscope, the mucosal tissue of interest may not just be electrically interrogated but also visually inspected.

The present disclosure has described one or more preferred embodiments, and it should be appreciated that many equivalents, alternatives, variations, and modifications, aside from those expressly stated, are possible and within the scope of the invention.

What is claimed is:

1. A mucosal impedance measuring device for measuring a pressure-controlled impedance of mucosa, the device comprising:
an endoscope having an elongated body extending to a scope end in which the scope end is articulable relative to the elongated body; and
a plurality of impedance measuring electrodes supported by the endoscope proximate the scope end;
wherein, upon articulation of the scope end, the plurality of impedance measuring electrodes are moved relative to the elongated body to be drawn into contact with mucosa under an applied pressure.

2. The mucosal impedance measuring device of claim 1, further comprising an impedance measuring system in electrical communication with the plurality of impedance measuring electrodes in which the impedance measuring system is configured to direct a current between the plurality of impedance measuring electrodes and through the mucosa and to measure the pressure-controlled impedance of the mucosa.

3. The mucosal impedance measuring device of claim 2, wherein the impedance measuring system includes software configured to determine whether the pressure-controlled impedance of the mucosa is a stable impedance measurement indicative of consistent pressure-regulated contact between the plurality of impedance measuring electrodes and the mucosa.

4. The mucosal impedance measuring device of claim 3, wherein the articulation of the scope end of the endoscope is manually controlled by an operator.

5. The mucosal impedance measuring device of claim 1, further comprising a support attachable to the endoscope proximate the scope end that supports the plurality of impedance measuring electrodes thereon.

6. The mucosal impedance measuring device of claim 5, wherein the support is a sleeve that at least partially surrounds the circumference of the elongated body of the endoscope at the scope end.

7. The mucosal impedance measuring device of claim 6, wherein the support is tubular.

8. The mucosal impedance measuring device of claim 6, wherein the support is compressively connected to the scope end of the endoscope.

9. The mucosal impedance measuring device of claim 1, further comprising a plurality of conductors in which each of the conductors being in electrical communication with a corresponding one of the plurality of impedance measuring electrodes and in which the plurality of conductors extend from the impedance measuring electrodes.

10. The mucosal impedance measuring device of claim 9, wherein the plurality of conductors are wires.

11. The mucosal impedance measuring device of claim 9, wherein the plurality of conductors are received in a working channel of the endoscope.

12. The mucosal impedance measuring device of claim 9, wherein the plurality of conductors extend to a connector on an end of the conductors opposite their attachment to the impedance measuring electrodes.

13. The mucosal impedance measuring device of claim 12, wherein the connector includes a plurality of axially-spaced contacts with each of the plurality of axially-spaced contacts being in electrical communication with one of the plurality of conductors and thereby in electrical communication with a corresponding one of the plurality of impedance measuring electrodes.

14. A method of measuring a pressure-controlled impedance of mucosa, the method comprising:
articulating a scope end of an endoscope relative to an elongated body of the endoscope to draw a plurality of impedance measuring electrodes on the scope end of the endoscope into contact with mucosa under an applied pressure from the articulating; and
conducting a current between the plurality of impedance measuring electrodes and through the mucosa and measuring the pressure-controlled impedance of the mucosa.

15. The method of claim 14, wherein the step of measuring the pressure-controlled impedance involves using an impedance measuring system in electrical communication with the plurality of impedance measuring electrodes in which the impedance measuring system directs the current between the plurality of impedance measuring electrodes and through the mucosa and measures the pressure-controlled impedance of the mucosa.

16. The method of claim 15, further comprising determining, using the impedance measuring system, whether the pressure-controlled impedance of the mucosa is a stable impedance measurement indicative of consistent pressure-regulated contact between the plurality of impedance measuring electrodes and the mucosa.

17. The method of claim 16, wherein the step of determining is performed by software.

18. The method of claim 17, wherein step of articulating the scope end of the endoscope is performed manually by an operator and the step of determining whether the pressure-controlled impedance of the mucosa is a stable impedance measurement indicative of consistent pressure-regulated contact between the plurality of impedance measuring electrodes and the mucosa is performed automatically.

19. The method of claim 14, further comprising the step of attaching a support proximate the scope end in which the support includes the plurality of impedance measuring electrodes thereon.

20. The method of claim 14, wherein the support is compressively connected to the scope end of the endoscope.

21. A method of visually indicating a portion of a gastrointestinal tract, the method comprising:
recording one or more spatial locations of an endoscope during an insertion of the endoscope into and through the gastrointestinal tract; and
visually depicting a location of the endoscope within a generated image of the gastrointestinal tract.

22. The method of claim 21, further comprising the steps of:
articulating a scope end of an endoscope relative to an elongated body of the endoscope to draw a plurality of impedance measuring electrodes on the scope end of the endoscope into contact with mucosa under an applied pressure from the articulating;
conducting a current between the plurality of impedance measuring electrodes and through the mucosa and measuring the pressure-controlled impedance of the mucosa; and
visually indicating the pressure-controlled impedance of the mucosa on the generated image of the gastrointestinal tract at a location at which the pressure-controlled impedance of the mucosa is taken.

23. The method of claim 22, further comprising the steps of
repetitively collecting pressure-controlled impedance of the mucosa at various locations in the gastrointestinal tract; and
visually indicating the pressure-controlled impedance of the mucosa at various locations in the gastrointestinal tract on the generated image of the gastrointestinal tract corresponding to the locations at which each respective pressure-controlled impedance of the mucosa is taken.

24. The method of claim 22, wherein the step of visually indicating the pressure-controlled impedance of the mucosa on the generated image of the gastrointestinal tract at a location at which the pressure-controlled impedance of the mucosa is taken involves associating and displaying a color associated with a value of the pressure-controlled impedance in a region associated the location at which the pressure-controlled impedance is taken.
